(19) United States
(12) Patent Application Publication
(10) Pub. No.: US 2014/0200437 A1
(43) Pub. Date: Jul. 17, 2014

(54) DETECTION OF INTERNAL GAS LEAKAGE

(71) Applicant: EMPIRE TECHNOLOGY DEVELOPMENT LLC, Wilmington, DE (US)

(72) Inventors: Thomas A. Yager, Encinitas, CA (US); Alfred Yager, Carmel, CA (US)

(73) Assignee: Empire Technology Development LLC, Wilmington, DE (US)

(21) Appl. No.: 13/742,624

(22) Filed: Jan. 16, 2013

(51) Int. Cl. A61M 31/00; A61B 5/00; A61B 5/055

(52) U.S. Cl. CPC A61M 31/005; A61B 5/055; A61B 5/004; A61B 5/72
USPC 600/420

(57) ABSTRACT

A method placing a gas into a first cavity of a patient, where the gas includes hyperpolarized 3-Helium (3-He). At least a portion of the patient is imaged using MRI to detect the gas within the patient. Based at least in part on the imaging, a determination is made regarding whether at least a portion of the gas is present in a second cavity of the patient. Presence of the gas in the second cavity is indicative of a leakage of the first cavity.

### BACKGROUND

The following description is provided to assist the understanding of the reader. None of the information provided or references cited is admitted to be prior art.

A pneumoperitoneum refers to gas in the abdominal cavity of an individual. The gas that forms a pneumoperitoneum may originate from a leak in the rectum, large intestine, or small intestine of the individual. Such a leak can be referred to colorectal anastomotic leakage. The colorectal anastomotic leakage can occur as a result of a surgical procedure in which the rectum, colon, or an intestine is operated on. The leakage which causes a pneumoperitoneum can also be due to a perforated abdominal viscus, which may be caused by an ulcer, a tumor, or abdominal trauma. If it is not identified and treated quickly, a pneumoperitoneum can cause severe infection in the individual.

As noted above, the surgical procedure itself will produce pneumoperitoneum. A traditional method for detecting pneumoperitoneum involves placing an individual in a sitting upright position and using X-rays or a computed tomography (CT) scan to detect the pneumoperitoneum. However, it may take several days or weeks after colorectal surgery for a pneumoperitoneum caused by anastomotic leakage to be detected using a CT scan because approximately 5 cubic centimeters (cm³) of gas is required for detection by a CT scanner, and the gas is also dissolving into the blood stream. During the time that it takes for a 5 cm³ pneumoperitoneum to develop, the individual can develop severe infection due to the leakage. Another traditional method for detecting a pneumoperitoneum involves the use of barium in conjunction with X-rays or a CT scan. The barium is administered to the individual by an enema or having the individual drink a barium solution. However, if the barium leaks into the abdominal cavity, it may cause problems such as intestinal adhesions. Further, the use of barium requires some form of fasting and mechanical bowel preparation of the individual, which can be challenging for post-surgical patients.

### SUMMARY

An illustrative method includes placing a gas into a first cavity of a patient, where the gas includes 3-Helium (3-He). At least a portion of the patient is imaged to detect the gas within the patient. Based at least in part on the imaging, a determination is made regarding whether at least a portion of the gas is present in a second cavity of the patient. Presence of the gas in the second cavity is indicative of a leakage of the first cavity.

An illustrative apparatus includes a gas source, a tube, and a computing device. The gas source includes a gas, and the gas includes 3-Helium (3-He). A first end of the tube is connected to the gas source and a second end of the tube is configured to be inserted into a first cavity of a patient to deliver the gas into the first cavity. The computing device is configured to receive image data of at least a portion of the patient. The computing device is also configured to generate an image using the image data, wherein the image includes at least a portion of the first cavity, at least a portion of a second cavity of the patient, and the gas such that a determination can be made regarding whether at least a portion of the gas is present in the second cavity of the patient.

An illustrative system includes a gas source, a magnetic resonance imaging machine, and a computing device. The gas source includes a gas for insertion into a first cavity of a patient, where the gas includes 3-Helium (3-He). The magnetic resonance imaging machine is configured to generate image data of at least a portion of the patient. The computing device is configured to receive the image data. The computing device is also configured to generate an image using the image data, where the image includes at least a portion of the first cavity, at least a portion of a second cavity of the patient, and the gas such that a determination can be made regarding whether at least a portion of the gas is present in the second cavity of the patient.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 is a diagram illustrating a system for detecting colorectal leakage in accordance with an illustrative embodiment.

FIG. 2 is an example overlay image illustrating colorectal leakage in accordance with an illustrative embodiment.

FIG. 3 is a flow diagram illustrating a process for detecting colorectal leakage in accordance with an illustrative embodiment.

FIG. 4 is a block diagram illustrating a system for detecting colorectal leakage in accordance with an illustrative embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

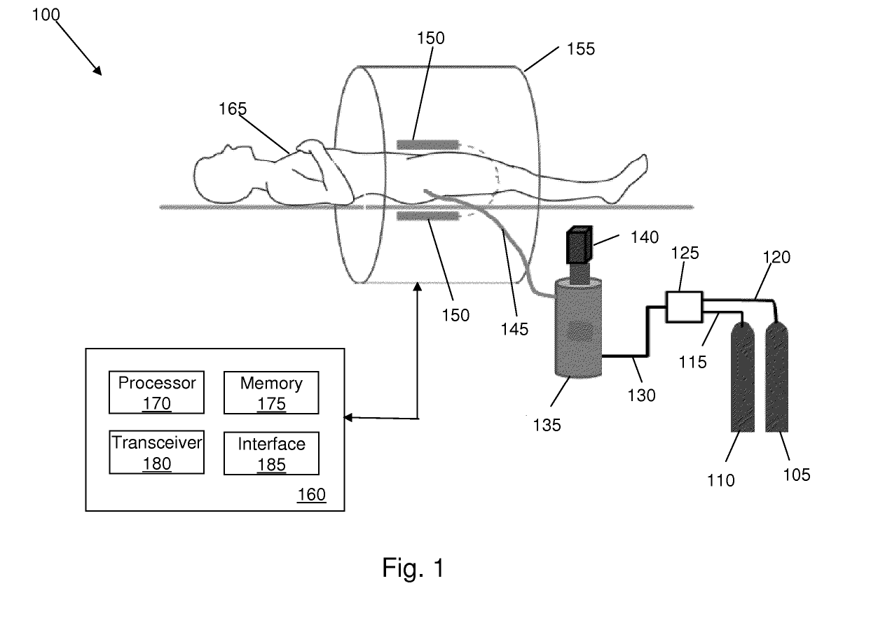

FIG. 1 is a diagram illustrating a system 100 for detecting colorectal leakage in accordance with an illustrative embodiment. System 100 includes a first gas source 105, a second gas source 110, a first gas outlet tube 115, a second gas outlet tube 120, a gas mixing chamber 125, a combined gas tube 130, a hyperpolarization chamber 135, a laser 140, a patient tube 145, coils 150, a magnetic resonance imaging (MRI) machine 155, and a computing system 160. In alternative embodiments, system 100 may include additional, fewer, and/or different elements. As discussed in detail below, system 100 is used to detect leakage of colorectal (or other) gas within a patient 165. As described herein, patient 165 is a human patient. In alternative embodiments, system 100 can be used to detect leakage of colorectal (or other) gas within animals such as livestock, pets, wild animals, etc.

In an illustrative embodiment, first gas source 105 includes 3-Helium (3-He), which is a non-radioactive and non-toxic isotope of helium that includes two protons and one neutron. The use of 3-He gas provides several advantages over traditional methods for detecting colorectal and other gas leakage. As described in more detail below, the 3-He gas used by system 100 can be hyperpolarized. Hyperpolarized 3-He is very selective and sensitive to nuclear magnetic resonance (NMR) imaging techniques. As a result, volumes as small as 0.03 cubic centimeters (cm³) of hyperpolarized 3-He can be resolved using NMR imaging techniques, and any detected hyperpolarized 3-He shows up with high contrast (i.e., visibility) in images obtained through NMR imaging techniques. The 3-He gas also has very low solubility in blood and other bodily fluids. As such, small quantities of 3-He that leak out of a cavity are detectable because the 3-He does not absorb into the patient's blood stream. In addition, helium can escape from very small openings in a cavity, which allows small leaks to be detected. As noted above, hyperpolarized 3-He is non-radioactive and non-toxic, and is therefore safe to place within a patient. Further, detection of 3-He within a patient can be done using NMR imaging techniques and does not require the use of harmful X-rays.

In an illustrative embodiment, second gas source 110 includes 4-Helium (4-He), which is a non-radioactive and non-toxic isotope of helium that includes two protons and two neutrons. In another illustrative embodiment, 4-He is used because it has a very low solubility in blood and surrounding tissue. In an alternative embodiment, second gas source 110 may include a different gas such as oxygen free carbon dioxide or nitrogen. The 3-He gas from first gas source 105 flows through first gas outlet tube 115 into gas mixing chamber 125, and the 4-He (or other) gas from second gas source 110 flows through second gas outlet tube 120 into gas mixing chamber 125. In an illustrative embodiment, gas mixing chamber 125 can be a receptacle that is configured to receive gas from first gas source 105 and second gas source 110 such that the two gases can mix within one another. Gas mixing chamber 125 may include a blower or circulation unit to help mix the two gases. Alternatively, gas mixing chamber 125 may be a receptacle that does not include a mechanism for actively mixing the two gases.

In one embodiment, the ratio of the 3-He gas to the 4-He (or other) gas in gas mixing chamber 125 is 50:50. In alternative embodiments, other ratios may be used such as 40:60, 30:70, 20:80, etc. In another alternative embodiment, the ratio may include more 3-He gas than 4-He (or other) gas. For example, the ratio of 3-He gas to 4-He (or other) gas may be 60:40, 70:30, 80:20, etc. The 4-He (or other) gas is used as a filler gas due to the high cost and limited availability of 3-He. In yet another alternative embodiment, second gas source 110 may not be used. In such an embodiment, the gas used by system 100 may be 100% 3-He, and gas mixing chamber 125 may not be used.

In an illustrative embodiment, the mixture of 3-He and 4-He flows from gas mixing chamber 125 through combined gas tube 130 and into hyperpolarization chamber 135. Hyperpolarization chamber 135 can be used to hyperpolarize the mixture of 3-He and 4-He gas to form a hyperpolarized gas mixture. Hyperpolarization can increase the detectability of 3-He up to a hundred thousand times. As such, using hyperpolarized 3-He improves the likelihood of detecting a leak because the hyperpolarized 3-He is more readily detectable than 3-He which has not been hyperpolarized. In one embodiment, hyperpolarization chamber 135 includes a polarized alkali metal vapor such as polarized Rubidium vapor (e.g., Rubidium-85 and/or Rubidium-87). In alternative embodiments, a different polarized alkali metal vapor may be used such as Sodium-23, Potassium-39, Cesium-133, etc.

The hyperpolarization can be performed using laser 140, which can be a high-power diode laser system. In one embodiment, rubidium atoms in hyperpolarization chamber 135 are optically pumped into one electronic spin state via circularly polarized light from laser 140, as known to those of skill in the art. The hyperpolarization of the 3-He and 4-He gas mixture is achieved by collisional spin exchange with the optically pumped rubidium atoms as known to those of skill in the art. The nuclear polarization achieved via collisional spin exchange is typically four to five orders of magnitude larger than the thermal equilibrium value of 3-He. As a result, the non-equilibrium state of hyperpolarization can be maintained for a period of time that is on the order of hours. However, once inserted into patient 165, the non-equilibrium state of hyperpolarization may last only on the order of minutes. As such, imaging is performed shortly after introduction of the 3-He into patient 165.

In an illustrative embodiment, the nuclei of the non-zero spin isotope 3-He is hyperpolarized to at least 10%. Alternatively, the hyperpolarization percentage may be less than 10%. In alternative embodiments, the 3-He and 4-He gas mixture may be hyperpolarized using any other method known to those of skill in the art. In one embodiment, the 3-He and 4-He gas mixture is hyperpolarized immediately prior to introduction of the gas mixture into patient 165. Alternatively, the 3-He and 4-He gas mixture may be hyperpolarized in advance and stored prior to introduction of the gas mixture into patient. In such an embodiment, the hyperpolarized gas can be stored and transported in tedlar bags.

The hyperpolarized 3-He and 4-He gas mixture flows from hyperpolarization chamber 135 into patient tube 145, and from patient tube 145 into patient 165. In an illustrative embodiment, patient tube 145 is inserted into the anus of patient 165 such that the hyperpolarized 3-He and 4-He gas mixture flows into the rectum and intestines of patient 165. In an alternative embodiment, patient tube 145 may be inserted into the mouth of patient 165, down the esophagus, into the stomach, and through the duodenum of patient 165 to test for gas leakage in the small intestine, stomach, etc. In an illustrative embodiment, the pressure with which the gas mixture is placed into patient 165 is controlled to prevent injury. In an illustrative embodiment, the gas pressure may be introduced at a pressure from 0-5 millimeters mercury (mmHg), but pressures up to approximately 40 mmHg can be administered with doctor supervision. In another illustrative embodiment, a volume up to approximately 1.0 liter of gas may be used if the gas is delivered near the area of concern, but up to approximately 8.0 liters of gas can be administered with doctor supervision. In one embodiment, the end of patient tube 145 which is inserted into patient 165 is inserted far enough into patient 165 such that leakage of the 3-He gas mixture into the surrounding environment is not a concern. Alternatively, a gas-tight seal can be formed at the entry point of patient tube 145 into patient 165 such that the hyperpolarized 3-He and 4-He gas mixture does not escape into the surrounding environment. The gas-tight seal can be formed using any method known to those of skill in the art. In one embodiment, a lotion or other lubricant may be used to insert patient tube 145 into patient 165, and the lubricant can help to form a seal between patient tube 145 and patient 165 that prevents the 3-He gas mixture from escaping through the anus or other entry point. In one embodiment, a helium sensor as known to those of skill in the art can be used to detect whether any of the 3-He gas mixture has leaked from the patient into the surrounding environment.

Once the hyperpolarized 3-He and 4-He gas mixture is placed into patient 165, imaging is performed using MRI machine 155 and coils 150. In an illustrative embodiment, MRI machine 155 can be a traditional MRI machine that is modified by the introduction of coils 150. In another illustrative embodiment, coils 150 can be a Helmholtz pair of radio frequency (RF) coils. The Helmholtz pair of radio frequency coils can operate at 48.5 mega-Hertz (MHz) and can have an element diameter of 28.5 centimeters (cm). The magnetic field used by MRI machine 155 can be 1.5 Tesla (T). In alternative embodiments, a different coil frequency, a different element diameter, and/or a different magnetic field strength may be used. The RF frequency for the resonance of hyperpolarized 3-He is proportional to the magnetic field strength. Some possible frequencies/magnetic field strengths of 93.9 MHz/3 T, 64.8 MHz/2 T, and 48.5 MHz/1.5 T can be used, but the selection is dependent on the magnetic field strength of the MRI equipment. Several antenna designs are feasible as known to those of skill in the art. These antenna geometries and impedance must support resonance at the RF frequency being used, while being capable of uniform irradiation over the volume of interest and able to fit within the MRI scanning magnet. The use of an approximately 48.5 MHz frequency at approximately 1.5 T allows MRI machine 155 to effectively couple to the 3-He nuclei to control the atomic spin of the nuclei.

As illustrated in FIG. 1, a first coil of coils 150 is placed above patient 165 and a second coil of coils 150 is placed below patient 165. In alternative embodiments, coils 150 may be placed on the sides of patient 165 instead of the front and back of patient 165. FIG. 1 illustrates coils 150 positioned about a mid-section of patient 165 such that the mid-section can be imaged. Coils 150 can alternatively be placed about a stomach area of patient 165, about a chest of patient 165, about an abdomen of patient 165, etc. A distance between the first and second coils can be based on the size of patient 165, with a larger distance between coils 150 being used for a larger patient and a smaller distance between coils 150 being used for a smaller patient.

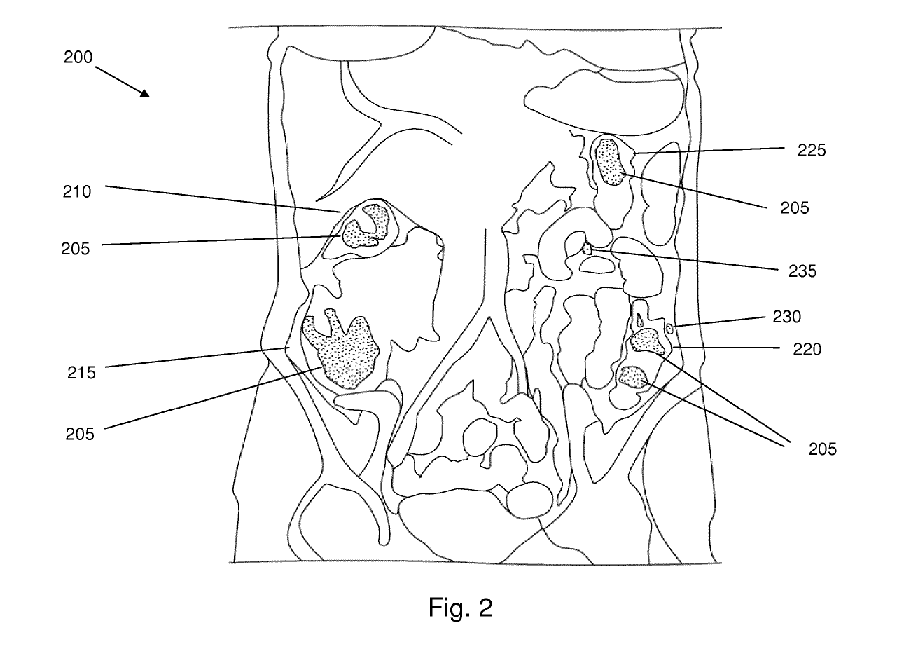

In an illustrative embodiment, MRI machine 155 and coils 150 are used to conduct a first MRI scan of patient 165. The first MRI scan can be used to detect any 3-He within patient 165. In another illustrative embodiment, MRI machine 155 is also used to conduct a second (regular) MRI scan of patient 165. As such, the first MRI scan can be used to generate an image that identifies any areas at which 3-He is detected in patient 165. The second MRI scan can be used to generate an image that illustrates the organs, organ walls, etc. within patient 165. Computing device 160 can be used to overlay the image in which 3-He is detected on top of the image that illustrates the organs, etc. within patient to generate an overlay image. As such, a physician can examine the overlay image to determine whether 3-He has leaked out of the rectum, large intestine, etc. into other areas of patient 165. An overlay image is described in more detail below with reference to FIG. 2.

The overlay image should illustrate 3-He within the rectum, colon, intestines, etc. The 3-He gas within these areas is not of interest. If there is any leakage within patient 165, the overlay image will illustrate 3-He in one or more areas outside of the rectum, colon, and intestines. The 3-He which has leaked may appear as one or more gas bubbles on a sterile side of the colon, rectum, intestines, etc. near the site of an anastomosis, near damaged tissue, near an ulcer, etc. The 3-He which leaks may also appear on the overlay image as a pneumoperitoneum in an abdomen of patient 165 or in a different sterile area within patient 165.

As illustrated in FIG. 1, computing device 160 includes a processor 170, a memory 175, a transceiver 180, and an interface 185. In alternative embodiments, computing device 160 may included additional, fewer, and/or different components. Computing device 160 can be any type of computer or computing system known to those of skill in the art. Processor 160, which can be any type of computer processor known to those of skill in the art, can be used to generate an overlay image based on received image data, to control MRI machine 155, to execute instructions stored in memory 175, etc. Memory 175, which can be any type of computer memory or memories known to those of skill in the art, can be used to store image data obtained from MRI machine 155, to store instructions to be executed by processor 170, to store patient information, etc.

Transceiver 180, which can include any type of receiver and/or transmitter known to those of skill in the art, can be used to communicate with MRI machine 155 and/or with other computing devices. Transceiver 180 can operate via wired or wireless communication according to any protocol(s) known to those of skill in the art. Transceiver 180 can receive image data from MRI machine 155 and processor 170 can be used to form images based on the image data. Transceiver 180 can also be used to provide instructions to MRI machine 155 such that an operator can control MRI machine 155 via interface 185. Interface 185 can be a user interface that allows a physician, technician, or other user to interact with computing device 160 and/or MRI machine 155. Interface 185 can include a keyboard, a mouse, a display, a touch screen, etc. In one embodiment, computing device 160 may be part of MRI machine 155. In an alternative embodiment, computing device 160 may be a standalone device that is in communication with MRI machine 155. In addition to controlling and/or interfacing with MRI machine 155, computing device 160 may also be used to control the release of gas from first gas source 105 and second gas source 110, to control laser 140, to control the release of hyperpolarized gas from hyperpolarization chamber 135 into patient, etc.

FIG. 2 is an example overlay image 200 illustrating colorectal leakage in accordance with an illustrative embodiment. As described with reference to FIG. 1, overlay image 200 can be formed by overlaying an image of detected 3-He on top of a traditional MRI image that illustrates the internal organs of a patient. As illustrated in FIG. 2, there is 3-He gas 205 within a hepatic flexure 210 of the patient, within an ascending colon 215 of the patient, within a colon 220 of the patient, and within a descending colon 225 of the patient. The 3-He gas 205 is expected because it was inserted into the rectum of the patient as described with reference to FIG. 1. However, overlay image 200 also illustrates 3-He gas 230 and 3-He gas 235 that has leaked out of the rectum. The 3-He gas 230 is indicative of a leak in colon 220, and 3-He gas 235 illustrates how gas that leaks from colon 220 may move away from colon 220 into other areas of the patient. Upon detection of the leak, the physician can perform surgery or take other corrective measures as known to those of skill in the art to prevent further leakage which may lead to severe infection.

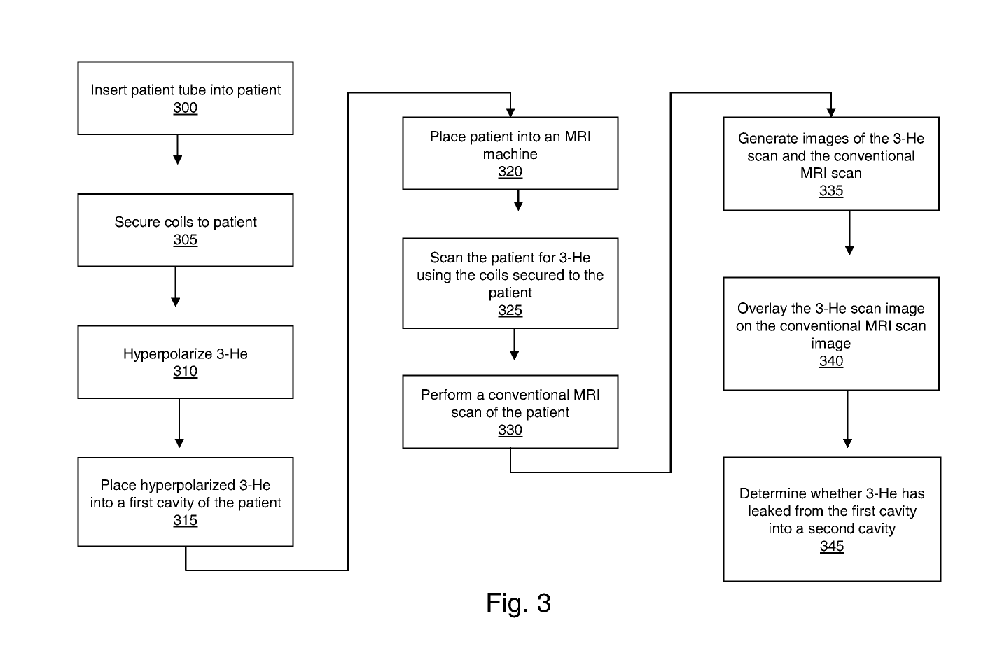

FIG. 3 is a flow diagram illustrating a process for detecting colorectal leakage in accordance with an illustrative embodiment. In alternative embodiments, fewer, additional, and/or different operations may be performed. Also, the use of a flow diagram is not meant to be limiting with respect to the order of operations performed. In an operation 300, a patient tube is inserted into a patient. In an illustrative embodiment, the patient tube is patient tube 145 described with reference to FIG. 1. In another illustrative embodiment, a first end of the patient tube is inserted into an anus of the patient such that hyperpolarized 3-He gas can be placed into the rectum of the patient. The first end of the patient tube can also be secured to the patient and/or sealed such that the 3-He gas is unable to escape into the environment. In alternative embodiments, the first end of the patient tube may be placed into and secured to any other orifice of the patient, such as a mouth or surgical opening of the patient that is to receive the 3-He gas. The second end of the patient tube can be in fluid communication with a supply of hyperpolarized 3-He.

In an operation 305, coils are secured to the patient. The coils can be secured using straps or any other method known to those of skill in the art. The coils can be coils 150 as described with reference to FIG. 1 (i.e., a Helmholtz pair of coils that operate at 48.5 MHz). Alternatively, any other radio frequency coils that are capable of being used to detect 3-He may be used. In an illustrative embodiment, a first coil is placed on a back side of the patient, or on the bottom of the patient when he/she is lying on his/her back on an MRI table. The second coil can be placed on a front side of the patient, or on the top of the patient when he/she is lying on his/her back on the MRI table. Alternatively, the coils may be placed on the sides of the patient. In another illustrative embodiment, the coils are placed directly across from one another such that the coils line up when secured to the patient. The coils can be placed about a first cavity of the patient that is to receive the hyperpolarized 3-He and/or over a second cavity of the patient into which the hyperpolarized 3-He gas may have leaked.

In an operation 310, the 3-He gas is hyperpolarized. The hyperpolarization may be performed using the method and system described with reference to FIG. 1. Alternatively, any other method known to those of skill in the art to hyperpolarize 3-He may be used. In one embodiment, the hyperpolarized 3-He that is used may have been hyperpolarized in advance of the procedure to test for leakage, and stored in a container until the procedure. In an illustrative embodiment, the 3-He gas may be mixed with 4-He or another gas as described with reference to FIG. 1. Alternatively, the gas used may be pure 3-He.

In an operation 315, the hyperpolarized 3-He is placed into the first cavity of the patient using the patient tube that is secured to the patient. In one embodiment, the hyperpolarized 3-He may be pressurized and the pressure can be used to move the hyperpolarized 3-He from the source container through the patient tube and into the patient. Alternatively, a pump may be used to move the hyperpolarized 3-He. In one embodiment, placement of the hyperpolarized 3-He into the patient may be controlled by a computing device such as computing device 160 described with reference to FIG. 1. Alternatively, placement of the hyperpolarized 3-He into the patient may be manually performed by a physician or other operator.

In an operation 320, the patient is placed into an MRI machine. The patient may be placed into the MRI machine before or after the patient tube and/or coils are secured to the patient. The MRI machine can be any MRI machine known to those of skill in the art that is capable of being used to detect hyperpolarized 3-He as described herein. In an operation 325, the patient is scanned for 3-He using the MRI machine and the coils secured to the patient. In an illustrative embodiment, the 3-He scan is performed as described with reference to FIG. 1. In an operation 330, a conventional MRI scan of the patient is performed. The conventional MRI scan can be performed by any method known to those of skill in the art. The conventional MRI scan may occur prior to or after the 3-He scan, depending on the embodiment.

In an operation 335, images of the 3-He scan and of the conventional MRI scan are generated. In an illustrative embodiment, the images are generated using a computing device such as computing device 160 described with reference to FIG. 1. The images may be generated by a computer that is part of the MRI machine or a computer that is remote from the MRI machine, depending on the embodiment. The images can be generated using any methods known to those of skill in the art. In an operation 340, the 3-He scan image is overlaid onto the conventional MRI scan image and an overlay image is generated. In an illustrative embodiment, the overlay image is generated using a computing device such as computing device 160 described with reference to FIG. 1. In an alternative embodiment, the overlay image may not be generated and solely the 3-He scan image may be used to identify any leaking 3-He gas. In an operation 345, a determination is made regarding whether any of the 3-He gas has leaked from the first cavity into a second cavity of the patient. In one embodiment, the determination is made by a physician or other user manually analyzing the overlay image. Alternatively, the determination may be made by a computing device such as computing device 160. If any of the 3-He has leaked into the second cavity, the physician can take appropriate measures to seal the leak and prevent infection in the patient.

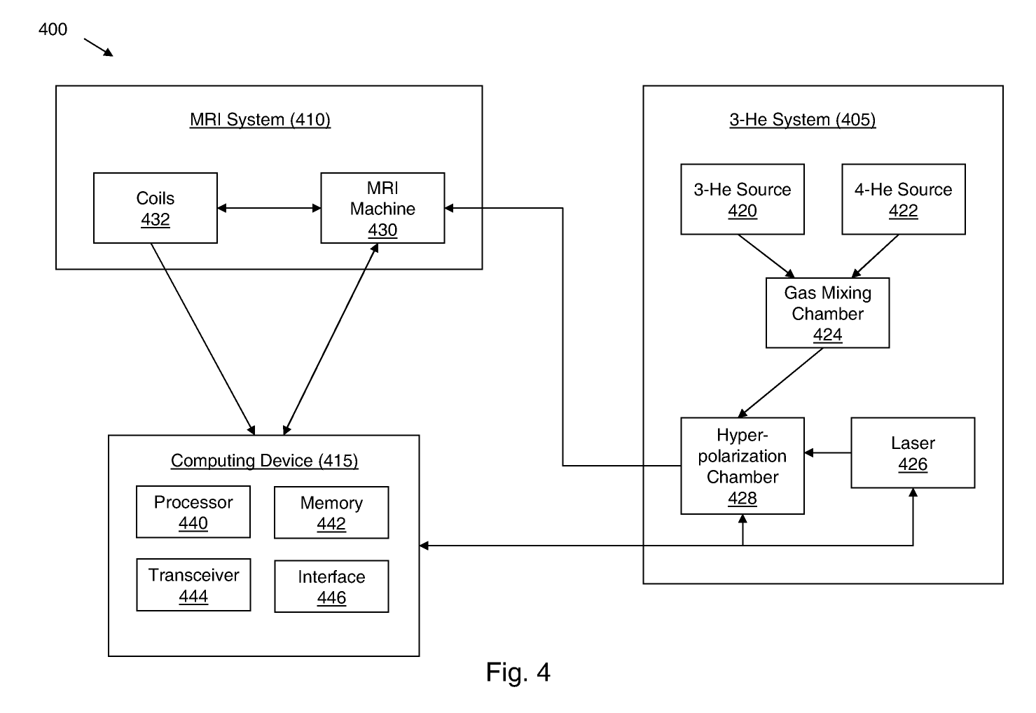

FIG. 4 is a block diagram illustrating a system 400 for detecting colorectal leakage in accordance with an illustrative embodiment. System 400 includes a 3-He system 405, an MRI system 410, and a computing system 415. In alternative embodiments, system 400 may include additional, fewer, and/or different elements. In an illustrative embodiment, 3-He system 405 includes a 3-He source 420, a 4-He source 422, a gas mixing chamber 424, a laser 426, and a hyperpolarization chamber 428. MRI system 410 includes an MRI machine 430 and coils 432. Computing device 415 includes a processor 440, a memory 442, a transceiver 444, and an interface 446. In alternative embodiments, 3-He system 405, MRI system 410, and/or computing device 415 may include additional, fewer, and/or different elements.

Computing device 415 can be any type of computer or computing system known to those of skill in the art. Processor 440, which can be any type of computer processor known to those of skill in the art, can be used to process received data, generate images, control MRI system 410, and/or control 3-He system 405 as described herein. Memory 442, which can be any type of computer memory or memories known to those of skill in the art, can be used to store data received from MRI system 410, to store data received from 3-He system 405, to store instructions to be executed by processor 440, to store patient information, etc. Transceiver 444, which can include any type of receiver and/or transmitter known to those of skill in the art, can be used to communicate with MRI system 410, 3-He system 405, and/or with other computing devices. Transceiver 180 can operate via wired or wireless communication according to any protocol(s) known to those of skill in the art. Interface 446 can be a user interface that allows a physician, technician, or other user to interact with computing device 415, MRI system 410, and 3-He system 405. Interface 446 can include a keyboard, a mouse, a display, a touch screen, etc.

In an illustrative embodiment, 3-He source 420 includes 3-Helium (3-He) gas as described herein. In another illustrative embodiment, 4-He source 422 includes 4-Helium (4-He) gas, as described herein. In an alternative embodiment, 4-He source 422 may not be used, or a gas other than 4-He such as oxygen free carbon dioxide or nitrogen may be used. The 3-He gas from 3-He source 420 flows through a gas outlet tube into gas mixing chamber 424, and the 4-He gas from 4-He source 422 flows through a gas outlet tube into gas mixing chamber 424. Release of the 3-He gas and 4-He gas may be controlled by processor 440 of computing device 415 to ensure that the desired volume of each gas is released. Alternatively, the release of gas may be manually performed by a user of system 400. In an illustrative embodiment, gas mixing chamber 424 can be a receptacle that is configured to receive 3-He gas and 4-He gas such that the two gases can mix within one another. Gas mixing chamber 424 may include a blower or circulation unit to help mix the two gases. Alternatively, gas mixing chamber 424 may be a receptacle that does not include a mechanism for actively mixing the two gases.

In one embodiment, the ratio of the 3-He gas to the 4-He gas in gas mixing chamber 424 is 50:50. In alternative embodiments, other ratios may be used as described herein. The mixture of 3-He gas and 4-He gas flows from gas mixing chamber 424 through a combined gas tube and into hyperpolarization chamber 428. Hyperpolarization chamber 428 can be used to hyperpolarize the mixture of 3-He and 4-He gas to form a hyperpolarized gas mixture as described herein. In one embodiment, hyperpolarization chamber 428 includes a polarized alkali metal vapor such as polarized Rubidium vapor (e.g., Rubidium-85 and/or Rubidium-87). In alternative embodiments, a different polarized alkali metal vapor may be used such as Sodium-23, Potassium-39, Cesium-133, etc.

The hyperpolarization can be performed using laser 426, which can be a high-power diode laser system. In one embodiment, rubidium atoms in hyperpolarization chamber 428 are optically pumped into one electronic spin state via circularly polarized light from laser 426, as known to those of skill in the art. Laser 426 can be controlled by computing device 415. Alternatively, laser 426 may be manually controlled by a user of system 400. The hyperpolarization of the 3-He and 4-He gas mixture is achieved by collisional spin exchange with the optically pumped rubidium atoms as known to those of skill in the art. In an illustrative embodiment, the nuclei of the non-zero spin isotope 3-He is hyperpolarized to at least 10%. Alternatively, the hyperpolarization percentage may be less than 10.

The hyperpolarized 3-He and 4-He gas mixture flows from hyperpolarization chamber 428 into the patient in MRI machine 430 through a patient tube. In an illustrative embodiment, the patient tube is inserted into the anus of the patient such that the hyperpolarized 3-He and 4-He gas mixture flows into the rectum and intestines of the patient. In an alternative embodiment, the patient tube may be inserted into the mouth of the patient, down the esophagus, into the stomach, and through the duodenum of the patient. In an illustrative embodiment, the pressure with which the gas mixture is placed into the patient is controlled to prevent injury. The pressure can be controlled by computing device 415. Alternatively, the pressure may be manually controlled by a user of system 400. In an illustrative embodiment, the gas pressure may be introduced at a pressure from 0-5 millimeters mercury (mmHg), but pressures up to approximately 40 mmHg can be administered with doctor supervision. In another illustrative embodiment, a volume up to approximately 1.0 liter of gas may be used if the gas is delivered near the area of concern, but up to approximately 8.0 liters of gas can be administered with doctor supervision. In one embodiment, the volume of gas inserted into the patient is monitored and controlled by computing device 415.

Once the hyperpolarized 3-He and 4-He gas mixture is placed into the patient, imaging is performed using MRI machine 430 and coils 432. In an illustrative embodiment, MRI machine 430 can be a traditional MRI machine that is modified by the introduction of coils 432. In another illustrative embodiment, coils 432 can be a Helmholtz pair of radio frequency (RF) coils. The Helmholtz pair of radio frequency coils can operate at 48.5 mega-Hertz (MHz) and can have an element diameter of 28.5 centimeters (cm). The magnetic field used by MRI machine 432 can be 1.5 Tesla (T). In alternative embodiments, a different coil frequency, a different element diameter, and/or a different magnetic field strength may be used. In an illustrative embodiment, a first coil of coils 432 is placed above the patient and a second coil of coils 432 is placed below the patient to cover an area of interest with respect to gas leakage. In alternative embodiments, coils 432 may be placed on the sides of the patient.

In an illustrative embodiment, MRI machine 430 and coils 432 are used to conduct a first MRI scan of the patient. The first MRI scan can be used to detect any 3-He within the patient using coils 432. MRI machine 430 may be controlled using transceiver 444 and processor 440 of computing device 415 through a wired or wireless connection, depending on the embodiment. Alternatively, MRI machine 430 may be a standalone unit with its own control system. Data regarding the first MRI scan can be provided from coils 432 and/or MRI machine 430 to transceiver 444 of computing device 415 for storage in memory 442 and analysis by processor 440 as described herein. In another illustrative embodiment, MRI machine 430 is also used to conduct a second (regular) MRI scan of the patient. As such, the first MRI scan can be used to generate an image that identifies any areas at which 3-He is detected in the patient and the second MRI scan can be used to generate an image that illustrates the organs, organ walls, etc. within the patient. Data regarding the second MRI scan can be provided from MRI machine 430 to transceiver 444 of computing device 415 for storage in memory 442 and analysis by processor 440.

Processor 440 of computing device 415 can be used to generate first and second images based on the data received from the first MRI scan and the second MRI scan of the patient, respectively. Processor 440 can also be used to generate an overlay image in which the first image (corresponding to detected 3-He gas) is overlaid on the second image (which illustrates the organs, etc. within the patient). As such, a physician can examine the overlay image to determine whether 3-He has leaked out of the rectum, large intestine, etc. into other areas of the patient. The first image, the second image, and/or the overlay image can be displayed and/or manipulated by a user through interface 446 of computing device 415. The images can also be saved in memory 442 and/or transmitted to another device by transceiver 444. If there is any leakage within the patient, the overlay image will illustrate 3-He in one or more areas outside of the rectum, colon, intestines, and other organs of the patient. Upon detection of the 3-He, the physician can take appropriate action to eliminate the leak and prevent subsequent leakage.

The systems and processes described herein provide significant advantages over traditional methods of detecting colorectal leakage. The embodiments described herein can be used to detect leakages that result from surgeries such as anastomoses for colon cancer, rectal cancer, Crohn's disease, ulcerative colitis, etc. The disclosed embodiments can also detect leakages due to diverticulitis, colon trauma, tumors, ulcers, other diseases of the colon, etc. The use of 3-He gas provides greater sensitivity (and thus detectability) over traditionally used materials such as barium. The 3-He gas is also able to detect smaller leaks than traditional methods because helium escapes more easily than other gases due to its composition. The 3-He gas also has low solubility in blood. As such, 3-He which leaks does not dissipate and can be detected to identify a leak. Also, 3-He is detected using an MRI and does not involve the use of harmful X-rays. The 3-He also does not require fasting or mechanical bowel preparation as is done when barium is used. Further, 3-He is non-toxic and does not require introducing any toxic chemicals into the patient. In addition, it is well known that rectal and other surgeries may produce a pneumoperitoneum that is the result of the surgery itself and not the result of colorectal leakage. Traditional methods for detecting leakage require that the pneumoperitoneum dissipates (i.e., dissolves into the blood stream of the patient) before being able to detect whether there is leakage. The use of 3-He allows for immediate detection of a leak because the 3-He can be distinguished from an existing pneumoperitoneum using the imaging techniques described herein.

Any of the operations described herein can be performed by computer-readable (or computer-executable) instructions that are stored on a computer-readable medium such as memory 175 or memory 442. The computer-readable medium can be a computer memory, database, or other storage medium that is capable of storing such instructions. Upon execution of the computer-readable instructions by a computing device such as computing device 160 or computing device 415, the instructions can cause the computing device to perform the operations described herein.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A method comprising:
placing a gas into a first cavity of a patient, wherein the gas includes 3-Helium (3-He);
imaging at least a portion of the patient to detect the gas within the patient; and
determining, based at least in part on the imaging, whether at least a portion of the gas is present in a second cavity of the patient, wherein presence of the gas in the second cavity is indicative of a leakage of the first cavity.

2. The method of claim 1, wherein placing the gas into the first cavity of the patient comprises placing a tube into the first cavity, and wherein the gas flows through the tube.

3. The method of claim 1, wherein the gas comprises hyperpolarized 3-He.

4. The method of claim 1, wherein the gas comprises a one to one ratio of 3-He and 4-He.

5. The method of claim 1, wherein the imaging is performed by way of nuclear magnetic resonance.

6. The method of claim 1, wherein the imaging is performed with a magnetic resonance imaging device.

7. The method of claim 1, wherein the first cavity comprises a large intestine of the patient, and wherein the second cavity is formed at least in part by an abdomen of the patient.

8. An apparatus comprising: a gas source that includes a gas, wherein the gas includes 3-Helium (3-He);
a tube, wherein a first end of the tube is connected to the gas source and a second end of the tube is configured to be inserted into a first cavity of a patient to deliver the gas into the first cavity; and
a computing device, wherein the computing device is configured to:
receive image data of at least a portion of the patient; and
generate an image using the image data, wherein the image includes at least a portion of the first cavity, at least a portion of a second cavity of the patient, and the gas such that a determination can be made regarding whether at least a portion of the gas is present in the second cavity of the patient.

9. The apparatus of claim 8, wherein the gas comprises hyperpolarized 3-He.

10. The apparatus of claim 8, wherein the gas comprises a one to one ratio of 3-He and 4-He.

11. The apparatus of claim 8, wherein the image data is obtained by way of nuclear magnetic resonance.

12. The apparatus of claim 8, further comprising a magnetic resonance imaging device configured to generate the image data.

13. The apparatus of claim 8, wherein the first cavity comprises a large intestine of the patient, and wherein the second cavity is formed at least in part by an abdomen of the patient.

14. The apparatus of claim 8, further comprising a laser configured to polarize the 3-He by way of collisional spin exchange.

15. The apparatus of claim 14, wherein the 3-He is polarized to greater than ten percent.

16. A system comprising:
a gas source that includes a gas for insertion into a first cavity of a patient, wherein the gas includes 3-Helium (3-He);
a magnetic resonance imaging machine configured to generate image data of at least a portion of the patient; and
a computing device, wherein the computing device is configured to:
receive the image data; and
generate an image using the image data, wherein the image includes at least a portion of the first cavity, at least a portion of a second cavity of the patient, and the gas such that a determination can be made regarding whether at least a portion of the gas is present in the second cavity of the patient.

17. The system of claim 16, further comprising a pair of radio frequency (RF) coils for use within the magnetic resonance imaging machine, wherein a first coil of the pair of RF coils is configured to be placed on a front side of the patient and a second coil of the pair of RF coils is configured to be placed on a rear side of the patient.

18. The system of claim 17, wherein the pair of radio frequency coils operate at a frequency of 48.5 megahertz.

19. The system of claim 16, wherein the processing unit is further configured to determine, based at least in part on the image data, whether the gas is present within the second cavity of the patient.

20. The system of claim 16, further comprising a laser configured to polarize the 3-He by way of collisional spin exchange.
